SEQUENCE LISTING (51 sequences)

SEQ ID NO: 1 - protein, 25 aa, synthetic construct
HLSLRGLPVCAFSSAGPCALRFTSA

SEQ ID NO: 2 - protein, 25 aa, synthetic construct
LSAMSTTDLEAYFKDCLFKDWEELG

SEQ ID NO: 3 - protein, 36 aa, synthetic construct
VLSRKYTSFPWLLGCTANWILRGTSFVYVPSALNPA

SEQ ID NO: 4 - protein, 25 aa, synthetic construct
AARLCCQLDPARDVLCLRPVGAESR

SEQ ID NO: 5 - protein, 39 aa, synthetic construct
VVNHYFQTRHYLHTLWKAGILYKRETTRSASFCGSPYSW

SEQ ID NO: 6 - protein, 39 aa, synthetic construct
YPALMPLYACIQAKQAFTFSPTYKAFLSKQYMNLYPVAR

SEQ ID NO: 7 - protein, 25 aa, synthetic construct
DKCLKFYSKISEYRHYCYSLYGTTL

SEQ ID NO: 8 - protein, 32 aa, synthetic construct
RCINCQKPLCPEEKQRHLDKKQRFHNIRGRWT

SEQ ID NO: 9 - protein, 35 aa, synthetic construct
MHGDTPTLHEYMLDLQPETTDLYCYEQLNDSSEEE

SEQ ID NO: 10 - protein, 35 aa, synthetic construct
LYCYEQLNDSSEEEDEIDGPAGQAEPDRAHYNIVT

SEQ ID NO: 11 - protein, 35 aa, synthetic construct
TLRLCVQSTHVDIRTLEDLLMGTLGIVCPICSQKP

SEQ ID NO: 12 - protein, 32 aa, synthetic construct
MHQKRTAMFQDPQERPRKLPQLCTELQTTIHD

SEQ ID NO: 13 - protein, 32 aa, synthetic construct
LPQLCTELQTTIHDIILECVYCKQQLLRREVY

SEQ ID NO: 14 - protein, 25 aa, synthetic construct
KQQLLRREVYDFAFRDLCIVYRDGN

SEQ ID NO: 15 - protein, 26 aa, synthetic construct
RDLCIVYRDGNPYAVCDKCLKFYSKI

SEQ ID NO: 16 - protein, 25 aa, synthetic construct
HYCYSLYGTTLEQQYNKPLCDLLIR

SEQ ID NO: 17 - protein, 32 aa, synthetic construct
YGTTLEQQYNKPLCDLLIRCINCQKPLCPEEK

SEQ ID NO: 18 - protein, 32 aa, synthetic construct
DKKQRFHNIRGRWTGRCMSCCRSSRTRRETQL

SEQ ID NO: 19 - protein, 9 aa, synthetic construct
RLCCQLDPA

SEQ ID NO: 20 - protein, 9 aa, synthetic construct
VLCLRPVGA

SEQ ID NO: 21 - protein, 10 aa, synthetic construct
GLPVCAFSSA

SEQ ID NO: 22 - protein, 9 aa, synthetic construct
GPCALRFTS

SEQ ID NO: 23 - protein, 9 aa, synthetic construct
LPVCAFSSA

SEQ ID NO: 24 - protein, 9 aa, synthetic construct
CLFKDWEEL

SEQ ID NO: 25 - protein, 10 aa, synthetic construct
DLEAYFKDCL

SEQ ID NO: 26 - protein, 8 aa, synthetic construct
ASFCGSPY

SEQ ID NO: 27 - protein, 10 aa, synthetic construct
ASFCGSPYSW

SEQ ID NO: 28 - protein, 10 aa, synthetic construct
RSASFCGSPY

SEQ ID NO: 29 - protein, 9 aa, synthetic construct
SFCGSPYSW

SEQ ID NO: 30 - protein, 9 aa, synthetic construct
ALMPLYACI

SEQ ID NO: 31 - protein, 7 aa, synthetic construct
  MOD_RES 4: note = X at position 4 is a cysteinylated cysteine residue
FKDXLFK

SEQ ID NO: 32 - protein, 12 aa, synthetic construct
LSAMSTTDLEAY

SEQ ID NO: 33 - protein, 6 aa, synthetic construct
DWEELG

SEQ ID NO: 34 - protein, 5 aa, synthetic construct
  MOD_RES 1: note = S at position 1 is modified so as to create U-Pam-14
SKKKK

SEQ ID NO: 35 - protein, 5 aa, synthetic construct
  MOD_RES 1: note = S at position 1 is modified so as to create U-Pam-12
SKKKK

SEQ ID NO: 36 - protein, 34 aa, synthetic construct
ADCCAKQEPERNECFLQHKDDNPNLPRLVRPEVD

SEQ ID NO: 37 - protein, 33 aa, Homo sapiens
PTLAKFSPYLGQMINLRRLLLSHIHASSYISPE

SEQ ID NO: 38 - protein, 33 aa, Homo sapiens
LKAMVQAWPFTCLPLGVLMKGQHLHLETFKAVL

SEQ ID NO: 39 - protein, 25 aa, Homo sapiens
KEGACDELFSYLIEKVKRKKNVLRL

SEQ ID NO: 40 - protein, 35 aa, Homo sapiens
ALYVDSLFFLRGRLDQLLRHVMNPLETLSITNCRL

SEQ ID NO: 41 - protein, 32 aa, Homo sapiens
VLYPVPLESYEDIHGTLHLERLAYLHARLREL

SEQ ID NO: 42 - protein, 35 aa, Homo sapiens
VWTSPRRLVELAGQSLLKDEALAIAALELLPRELF

SEQ ID NO: 43 - protein, 32 aa, Homo sapiens
LLPRELFPPLFMAAFDGRHSQTLKAMVQAWPF

SEQ ID NO: 44 - protein, 35 aa, Homo sapiens
LPSLSHCSQLTTLSFYGNSISISALQSLLQHLIGL

SEQ ID NO: 45 - protein, 33 aa, Homo sapiens
LETFKAVLDGLDVLLAQEVRPRRWKLQVLDLRK

SEQ ID NO: 46 - protein, 35 aa, Homo sapiens
CLPLGVLMKGQHLHLETFKAVLDGLDVLLAQEVRP

SEQ ID NO: 47 - protein, 34 aa, Homo sapiens
WTVWSGNRASLYSFPEPEAAQPMTKKRKVDGLST

SEQ ID NO: 48 - protein, 33 aa, Homo sapiens
YIAQFTSQFLSLQCLQALYVDSLFFLRGRLDQL

SEQ ID NO: 49 - protein, 33 aa, Homo sapiens
LQCLQALYVDSLFFLRGRLDQLLRHVMNPLETL

SEQ ID NO: 50 - protein, 35 aa, Homo sapiens
VLSLSGVMLTDVSPEPLQALLERASATLQDLVFDE

SEQ ID NO: 51 - protein, 34 aa, Homo sapiens
SISISALQSLLQHLIGLSNLTHVLYPVPLESYED

(19) United States
(12) Patent Application Publication
(10) Pub. No.: US 2026/0200990 A1
(43) Pub. Date: Jul. 16, 2026

(54) MODIFIED LONG PEPTIDES SUITABLE FOR USE IN IMMUNISATION

(71) Applicant: ISABELLA PHARMA B.V., KC Haarlem (NL)

(72) Inventors: Brigitte Elisa Anna BURM, Oegstgeest (NL); Thomas Johannes Maria BEENAKKER, Oegstgeest (NL); Lucien Nathanaël LAMEIJER, Oegstgeest (NL); Esmé Teunisje Ida VAN DER GRACHT, Oegstgeest (NL)

(73) Assignee: ISABELLA PHARMA B.V., KC Haarlem (NL)

(21) Appl. No.: 19/120,959

(22) PCT Filed: Oct. 13, 2023
(86) PCT No.: PCT/EP2023/078470

§ 371 Date: Apr. 14, 2025

(30) Foreign Application Priority Data
Oct. 13, 2022 (EP) 22201252.8
Jul. 5, 2023 (EP) 23183514.1

(51) Int. Cl. C07K 14/02; A61K 39/00; A61K 39/29; A61K 39/39; A61P 31/20

(52) U.S. Cl. CPC C07K 14/02; A61K 39/292; A61K 39/39; A61P 31/20; A61K 2039/55511; A61K 2039/572

(57) ABSTRACT

The present invention relates to the field of immunology. In particular, it relates to novel long cysteine-containing immunogenic peptides, wherein the cysteine has been modified to yield a more stable product while preserving immunogenicity. The invention further relates to compositions and methods for treating diseases using the long immunogenic peptides of the invention.

### FIELD OF THE INVENTION

The present invention relates to the field of immunology. In particular, it relates to novel long cysteine-containing immunogenic peptides, wherein the cysteine has been modified. The invention further relates to compositions and methods for treating diseases using the long immunogenic peptides of the invention.

### BACKGROUND OF THE INVENTION

Immunotherapy based on synthetic long peptides (SLPs) has shown promising results in the treatment of cancer and other diseases. For example, an immunotherapeutic consisting of SLPs of the E6 and E7 oncoproteins of high-risk HPV16 induces potent CD4+ and CD8+ T-cell responses in patients with (pre-) malignant disease of the cervix and induces tumor regression in patients in combination with chemotherapy (Melief et al. 2022 Sci Trans Med 12:eaaz8235).

One of the main advantages of SLP-based immunotherapeutics as compared to recombinant proteins is that SLPs are produced synthetically rather than recombinantly. However, compositions of SLPs containing cysteines may be prone to multimer formation, in particular in an oxidizing environment, due to the formation of disulfide bonds between cysteines of two SLP molecules. Furthermore, SLPs containing two or more cysteine residues may form intramolecular disulfide bonds. Upon storage over time and/or in use, such multimer formation and/or intramolecular disulfide bond formation thus may result in loss (instability) of the desired monomeric SLP without intramolecular disulfide bonds. The present invention provides a solution for this problem by introducing cysteine modifications into the SLP that prevent formation of undesired disulfide bonds, while preserving immunogenicity.

### SUMMARY OF THE INVENTION

In a first main aspect, the invention relates to an immunogenic peptide of 20 to 45 amino acid residues in length comprising one or more cysteine residues, wherein said one or more cysteine residues is present in a modified form wherein said one or more cysteine residue is bound to a cysteine or other thiol-containing compound via a disulfide bond, wherein said disulfide bond is not an intramolecular disulfide bond between two cysteine residues within the same immunogenic peptide molecule and wherein said disulfide bond is also not an intermolecular disulfide bond connecting two immunogenic peptide molecules.

In a further aspect, the invention relates to an immunogenic composition or medicinal product comprising one or more immunogenic peptide according to the invention as described herein.

In further aspects, the invention relates to method of treatment, uses and method for the production of immunogenic peptides, immunogenic compositions or medicinal products of the invention.

### BRIEF DESCRIPTION OF THE FIGURES

FIG. 1: Part of the chemical structure of SEQ ID NO: 2 containing a cystine moiety (FKDC(C)LFK) (SEQ ID NO:31). The cysteine is cysteinylated and forms a disulfide bond with another cysteine molecule. R1=LSAMSTTDLEAY (SEQ ID NO:32), R2=DWEELG (SEQ ID NO:33).

FIG. 2: Chromatographic profiles of the two mixes analyzed at T=0. Each containing 6 different peptides which correspond to the codes in Table 3 and 4.

FIG. 3: Comparison of non-cysteine containing SLP, SLP-Cys-ox (only cysteine oxidation) and SLP-Cys/Met-ox (cysteine and methionine oxidation) in IFNy ELISpot. T cell reactivity towards SLP in human PBMCs from HBV resolvers and healthy donors. Shown is the number of spots per 150.000 cells induced after 4 day in vitro stimulation with individual SLPs (non-cysteine containing, SLP-Cys-ox or SLP-Cys/Met-ox) in combination with Amplivant. Background IFNy response, upon stimulation with Albumin control peptide, was subtracted from the spot count. Black and clear dots represent responses of HBV resolver PBMCs. Grey dots represent healthy controls. Positive IFNy responses, above healthy control responses, are indicated by clear symbols.

### DETAILED DESCRIPTION OF THE INVENTION

### Definitions

The term “immunogenic peptide”, when used herein, refers to a peptide capable of triggering or boosting an immune response, such as a local and/or systemic CD4+ and/or CD8+ T cell response and/or an antibody response. Likewise, the term “immunogenic composition” means a composition capable of triggering or boosting an immune response, such as a local and/or systemic CD4+ and/or CD8+ T cell response and/or an antibody response. Immunogenic peptides described herein, also denominated herein as long peptides, exceed the length of human leukocyte antigen (HLA) class I and class II presented ligands. Preferably, the long peptides of the invention are synthetic peptides, denominated herein as synthetic long peptides (SLPs).

In the context of describing peptides, the term “in length”, for example a peptide of 20 to 45 amino acids or amino acid residues “in length” refers to the number of amino acid residues in the linear peptide chain, i.e. not counting optional additional amino acid residues that are bound to cysteines in the peptide chain. For example, a peptide chain of 30 amino acids wherein one cysteine is present in the form of a cystine (cysteine dimer) is considered 30 amino acids in length for the purpose of the present invention.

The term “MHC class I ligand” refers to a peptide sequence that can bind to and be presented by an MHC class I molecule. MHC (major histocompatibility complex) class I molecules (in humans including HLA-A, HLA-B and HLA-C) are one of two classes of (MHC) molecules found on the cell surface of nucleated cells. Their function is to present peptide fragments of proteins to cytotoxic T cells, thus trigger an immune response. MHC class I ligands typically have a length of 8-11 amino acids. Proteins or long peptides, such as long peptides of the invention, that comprise MHC class I ligands typically require intracellular processing for the MHC class I ligand to be generated and made available to bind to the MHC Class I molecule and be presented on the cell surface. The intracellular processing typically occurs via proteasomal cleavage in the cytosol.

The term “MHC class II ligand” refers to a peptide sequence that can bind to and be presented by an MHC class II molecule. MHC (major histocompatibility complex) class II molecules (in humans including HLA-DP, HLA-DM, HLA-DOA, HLA-DOB, HLA-DQ, and HLA-DR) are one of two classes of (MHC) molecules found on the cell surface of antigen presenting cells. Their function is to present peptide fragments of proteins to T helper cells, thus trigger an immune response. MHC class II ligands typically have a length of 11-16 amino acids. Proteins or long peptides, such as long peptides of the invention, that comprise MHC class II ligands typically require intracellular processing for the MHC class II ligand to be generated and made available to bind to the MHC Class II molecule and be presented on the cell surface. The intracellular processing typically occurs via endocytosis and lysosomal digestion.

The term “CD8+ T cell response” refers to an immune response wherein cytotoxic CD8 expressing T cells are activated by a complex of an MHC class I molecule and a peptide ligand.

The term “immunization” in the context of medical treatment refers to the administration of an immunogenic peptide to a subject in order to trigger or boost an immune response.

The term “fragment” in the context of a protein refers to a sequence of consecutive amino acids that corresponds to, i.e. is identical to, a part of said protein sequence. This does not exclude, however, that the fragment may be further modified, e.g. conjugated, such as covalently bound to another molecule.

The term “corresponding” when used in connection with a sequence in the context of comparison of sequences, refers to the sequence with which a given sequence has the best alignment, as assessed with bioinformatic tools for alignment of sequences known in the art, such as BLAST.

Unless specified otherwise, the terms “treatment” and “treating”, when used herein in the context of a medical intervention, include both therapeutic treatment as well as prevention (prophylactic treatment).

“Therapeutic treatment” refers to the administration of an effective amount of an immunogenic peptide, an immunogenic composition or medicinal product with the purpose of easing, ameliorating, arresting or eradicating (curing) symptoms or disease states.

“Prevention” or “preventing” refers to the administration of an effective amount of an immunogenic peptide, an immunogenic composition or medicinal product with the purpose of preventing a disease.

An “effective amount” refers to an amount effective, at dosages and for periods of time necessary, to achieve a desired therapeutic result.

In the context of the present invention, the term “medicinal product” means a product for triggering or boosting an immune response. A medicinal product may be administered directly to a human subject or may be used in ex vivo immunization regimens. In ex vivo immunization regimens, the medicinal product may be used to generate antigen-loaded antigen presenting cells (APCs), such as antigen-loaded activated Dendritic Cells (DCs), and subsequently stimulate expansion of antigen-specific T cells (e.g. CD4 and CD8 positive circulating T cells, Tumor Infiltrating Lymphocytes (TILs)). Such antigen-loaded APCs or expanded antigen-specific T cells are subsequently administered to a human subject. A medicinal product may be a single immunogenic composition or comprise more than one immunogenic composition.

The term “oxidizing solvent” in the present context refers to a solvent wherein cysteine residues that are part of a peptide chain (and not modified according to the invention) would become oxidized over time, for example within 1 day, such as within 4 hours, for example within 1 hour.

### FURTHER ASPECTS AND EMBODIMENTS OF THE INVENTION

### Immunogenic Peptides

As described above, in a first main aspect, the invention relates to an immunogenic peptide of 20 to 45 amino acid residues in length comprising one or more cysteine residues, wherein said one or more cysteine residues is present in a modified form, wherein said one or more cysteine residue (i.e. all cysteine residues, if more than one is present) is/are bound to a cysteine or other thiol-containing compound via a disulfide bond, wherein said disulfide bond is not an intramolecular disulfide bond or intermolecular disulfide bond between immunogenic peptide molecules. 5 The cysteine modification prevents the formation of intermolecular and/or intramolecular disulfide bonds, resulting in a more stable peptide-based immunogenic composition or medicinal product. As shown in the Examples, the immunogenic peptides of the invention have retained their immunogenic properties in spite of the cysteine modification. As the peptides have a relatively long length 10 and thus require intracellular processing before the ligands contained within the peptides can be presented, the data suggest that the cysteine modifications do not affect intracellular processing.

The one or more cysteines in the immunogenic peptides of the invention have typically been modified such that the thiol group of the cysteine has been oxidized and thus forms a disulfide bond with another thiol-containing compound. Said other thiol-containing compound is not another cysteine residue in the same peptide or a cysteine residue in a different peptide molecule. Thus, no intramolecular bonds are formed within this peptide and no intermolecular disulfide bonds are formed between two or more immunogenic peptide molecules. I.e. there are no intermolecular disulfide bonds between two molecules of the same peptide (peptide molecules having the same sequence) and there are no intermolecular disulfide bonds between two different peptides (peptide molecules having a different sequence) if present in the same composition.

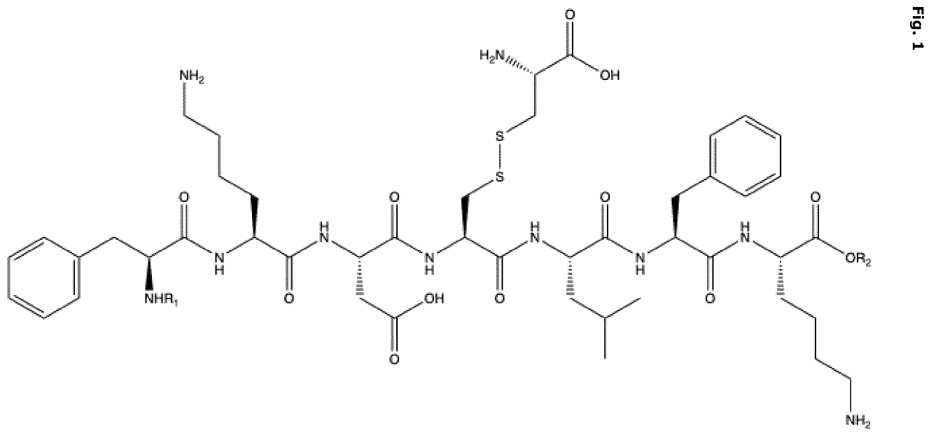

In one embodiment, said one or more modified cysteine residue is a cystine residue (L-cystine or D-cystine). Thus, in such an embodiment, the one or more cysteine residue in the immunogenic peptide is present in a form wherein the thiol group of the cysteine in the peptide has been oxidised (cysteinylated) and forms a disulfide bond with a cysteine molecule (the latter not being part of the immunogenic peptide). An example of such a peptide wherein the cysteine has been modified is 30 shown in FIG. 1.

In another embodiment, said one or more cysteine residue is bound, via a disulfide bond, to a compound selected from the group consisting of: glutathione, cysteinylglycine, homocysteine, y-glutamylcysteine, 2-mercaptoethanesulfonic acid, 2-mercaptoethanol, thioglycolic acid, acetylcysteine, cysteamine, (2S)-1-[(2S)-2-methyl-3-sulfanylpropanoyl]pyrrolidine-2-carboxylic acid, N-(methyl) mercaptoacetamide, 4-mercaptophenylacetic acid and 3-nitro-2-pyridinethiol.

In one embodiment, wherein said immunogenic peptide comprises two or more modified cysteine residues, such as three or more modified cysteine residues, for example four or more modified cysteine residues. If more than one cysteine is present in the peptide, they may all be modified or only some of them, such as only one or two may be modified.

If more than one cysteine is present in the peptide, they may all be modified in the same way, for example all be present in the form of cystine, or different types of modified cysteines may be used in one peptide.

In one embodiment, the immunogenic peptide does not comprise aminobutyric acid (Abu).

In one embodiment, if said immunogenic peptide contains a single modified cysteine residue, said modified cysteine residue is not the most C-terminal residue of said immunogenic peptide.

In one embodiment, immunogenic peptides of the invention do not have a cysteine residue at the N- or C-terminus of the peptide.

As mentioned, the immunogenic peptide is from 20 to 45 amino acids in length, i.e. it may be 20, 21, 22, 23, 24, 25, 26, 27, 28, 29, 30, 31, 32, 33, 34, 35, 36, 37, 38, 39, 40, 41, 42, 43, 44 or 45 amino acids in length.

In one embodiment wherein the peptide comprises from 20 to 25 amino acid residues, said peptide contains two or more of said modified cysteines.

In one embodiment, the immunogenic peptide is from 26 to 45 amino acid residues in length, such as 26, 27, 28, 29, 30, 31, 32, 33, 34, 35, 36, 37, 38, 39, 40, 41, 42, 43, 44 or 45 amino acids in length.

In one embodiment, the immunogenic peptide is from 27 to 45 amino acid residues in length, such as from 28 to 45, from 29 to 45, from 30 to 45, from 31 to 45, from 32 to 45, from 33 to 45, from 34 to 45 or from 35 to 45 amino acids in length.

In one embodiment, the immunogenic peptide is isolated, i.e. isolated, for example purified, from an environment containing other biomolecules, such as cellular components or non-peptide molecules, e.g. lipids or nucleic acids.

In one embodiment, the immunogenic peptide has been manufactured synthetically, for example using solid phase peptide synthesis.

In one embodiment, the immunogenic peptide further contains one or more methionine residues, wherein said methionine residues are not modified.

In another embodiment, the immunogenic peptide further contains one or more methionine residues, wherein said one or more methionine residues are present in the form a methionine sulfoxide.

In one embodiment, said immunogenic peptide comprises at least one MHC class I ligand. In one embodiment, the immunogenic peptide is a peptide that requires intracellular processing for the MHC class I ligand to be generated and made available to bind to the MHC Class I molecule. The resulting bound MHC class I ligands can be identified chemically, for example by mass spectrometry (MS) of peptides eluted from the relevant MHC class I molecule. Alternatively, the bound peptide can be detected functionally by T cells reactive with cells expressing this particular peptide-MHC class I combination. In one embodiment, the sequence of the MHC Class I ligand comprises one or more cysteine. In one embodiment, the one or modified cysteine residue is part of the MHC Class I ligand or within 10 amino acids, such as within 9, within 8, within 7, within 6, within 5, within 4, within 3, within 2 amino acids from either end of the MHC Class I ligand sequence, or immediately next to either end of the ligand sequence.

In one embodiment, said immunogenic peptide comprises at least one MHC class II ligand. MHC class II ligands can be identified chemically, for example by mass spectrometry (MS) of peptides eluted from the relevant MHC class II molecule. Alternatively, the bound peptide can be detected functionally by T cells reactive with cells expressing this particular peptide-MHC class II combination. In one embodiment, the sequence of the MHC Class II ligand comprises one or more cysteine. In one embodiment, the one or modified cysteine residue is part of the MHC Class II ligand or within 10 amino acids, such as within 9, within 8, within 7, within 6, within 5, within 4, within 3, within 2 amino acids from either end of the MHC Class II ligand sequence, or immediately next to either end of the ligand sequence.

In one embodiment, said immunogenic peptide comprises at least one MHC class I ligand and one MHC class II ligand.

In one embodiment, the immunogenic peptide of the invention is (except for the modification of the one or more cysteines and optionally methionines), a fragment of a known human protein, such as a fragment of a tumor protein or a fragment of a protein of a pathogen, such as a viral protein.

In one embodiment, the sequence of the immunogenic peptide corresponds to a fragment of a hepatitis B protein, such as X protein (X), polymerase (Pol) or HBcAg (core). In one embodiment, the sequence of the immunogenic peptide corresponds to a fragment of the hepatitis B X protein comprising one or more of the sequences set forth in SEQ ID NO:19-25. In another embodiment, the sequence of the immunogenic peptide corresponds to a fragment of the hepatitis B Pol protein comprising one or more of the sequences set forth in SEQ ID NO: 26-30.

In another embodiment, the sequence of the immunogenic peptide corresponds to a fragment of a human papillomavirus (HPV) antigen, such as E6 or E7, for example a fragment of HPV16 E6 or HPV16 E7.

In another embodiment, the sequence of the immunogenic peptide corresponds to a fragment of PRAME (preferentially expressed antigen in melanoma).

In one embodiment, the immunogenic peptide comprises or consists of a sequence selected from the group of sequences set forth in SEQ ID NO: 1-18 sequences or a sequence selected from the group of sequences set forth in SEQ ID 10 NO: 38, 39, 40 and 44.

TABLE 1
Sequence listing

| SEQ | | | Length |
|---|---|---|---|
| ID | Antigen | Sequence (AA) | (AA) |
| | HBV | | |
| 1 | X | HLSLRGLPVCAFSSAGPCALRFTSA | 25 |
| 2 | X | LSAMSTTDLEAYFKDCLFKDWEELG | 25 |
| 3 | Pol | VLSRKYTSFPWLLGCTANWILRGTSFVYVPSALNPA | 36 |
| 4 | X | AARLCCQLDPARDVLCLRPVGAESR | 25 |
| 5 | Pol | VVNHYFQTRHYLHTLWKAGILYKRETTRSASFCGSPYSW | 39 |
| 6 | Pol | YPALMPLYACIQAKQAFTFSPTYKAFLSKQYMNLYPVAR | 39 |
| | HPV16 | | |
| 7 | E6 | DKCLKFYSKISEYRHYCYSLYGTTL | 25 |
| 8 | E6 | RCINCQKPLCPEEKQRHLDKKQRFHNIRGRWT | 32 |
| 9 | E7 | MHGDTPTLHEYMLDLQPETTDLYCYEQLNDSSEEE | 35 |
| 10 | E7 | LYCYEQLNDSSEEEDEIDGPAGQAEPDRAHYNIVT | 35 |
| 11 | E7 | TLRLCVQSTHVDIRTLEDLLMGTLGIVCPICSQKP | 35 |
| 12 | E6 | MHQKRTAMFQDPQERPRKLPQLCTELQTTIHD | 32 |
| 13 | E6 | LPQLCTELQTTIHDIILECVYCKQQLLRREVY | 32 |
| 14 | E6 | KQQLLRREVYDFAFRDLCIVYRDGN | 25 |
| 15 | E6 | RDLCIVYRDGNPYAVCDKCLKFYSKI | 26 |
| 16 | E6 | HYCYSLYGTTLEQQYNKPLCDLLIR | 25 |
| 17 | E6 | YGTTLEQQYNKPLCDLLIRCINCQKPLCPEEK | 32 |
| 18 | E6 | DKKQRFHNIRGRWTGRCMSCCRSSRTRRETQL | 32 |
| | HBV | | |
| 19 | X | RLCCQLDPA | 9 |
| 20 | X | VLCLRPVGA | 9 |
| 21 | X | GLPVCAFSSA | 10 |
| 22 | X | GPCALRFTS | 9 |
| 23 | X | LPVCAFSSA | 9 |
| 24 | X | CLFKDWEEL | 9 |
| 25 | X | DLEAYFKDCL | 10 |
| 26 | Pol | ASFCGSPY | 8 |
| 27 | Pol | ASFCGSPYSW | 10 |
| 28 | Pol | RSASFCGSPY | 10 |
| 29 | Pol | SFCGSPYSW | 9 |
| 30 | Pol | ALMPLYACI | 9 |
| | PRAME | | |
| 37 | | PTLAKFSPYLGQMINLRRLLLSHIHASSYISPE | 33 |
| 38 | | LKAMVQAWPFTCLPLGVLMKGQHLHLETFKAVL | 33 |
| 39 | | KEGACDELFSYLIEKVKRKKNVLRL | 25 |
| 40 | | ALYVDSLFFLRGRLDQLLRHVMNPLETLSITNCRL | 35 |
| 41 | | VLYPVPLESYEDIHGTLHLERLAYLHARLREL | 32 |
| 42 | | VWTSPRRLVELAGQSLLKDEALAIAALELLPRELF | 35 |
| 43 | | LLPRELFPPLFMAAFDGRHSQTLKAMVQAWPF | 32 |
| 44 | | LPSLSHCSQLTTLSFYGNSISISALQSLLQHLIGL | 35 |
| 45 | | LETFKAVLDGLDVLLAQEVRPRRWKLQVLDLRK | 33 |
| 46 | | CLPLGVLMKGQHLHLETFKAVLDGLDVLLAQEVRP | 35 |
| 47 | | WTVWSGNRASLYSFPEPEAAQPMTKKRKVDGLST | 34 |
| 48 | | YIAQFTSQFLSLQCLQALYVDSLFFLRGRLDQL | 33 |
| 49 | | LQCLQALYVDSLFFLRGRLDQLLRHVMNPLETL | 33 |
| 50 | | VLSLSGVMLTDVSPEPLQALLERASATLQDLVFDE | 35 |
| 51 | | SISISALQSLLQHLIGLSNLTHVLYPVPLESYED | 34 |

### Peptides Used in the Compositions and Methods of the Invention

In some embodiments, the immunogenic peptides of the invention are capable of inducing a potent antigen-directed CD8+ cytotoxic T cell and/or CD4+T helper cell response, when administered to a human subject. The peptides may be predicted to be immunogenic and/or may be proven to be immunogenic using in vitro or ex vivo assays or by doing in vivo tests appreciated in the art to establish immunogenicity. Preferably, the peptides can be used effectively in the prevention, partial clearance and/or treatment or full clearance of a disease or condition in a subject, preferably as detectable by:
activation or an induction of the immune system and/or an increase in antigen-specific activated CD4+ and/or CD8+ T-cells in peripheral blood or in tissues as established by interferon-gamma ELISpot assay or by tetramer staining of CD4+ or CD8+ T cells or an increase of the cytokines (such as interferon-gamma, TNF-alpha, interleukin-2) produced by these T-cells as established by intracellular cytokine staining of CD4+ and CD8+ T cells in flow cytometry after at least one week of treatment; and/or
activation of an antibody response, for example with virus-neutralizing capacity and demonstrable in serum/plasma by ELISA and/or virus neutralization assay and/or

In a preferred embodiment, the medicinal product or (an) immunogenic composition(s) of the invention or used in the method of the invention comprise(s) a combination of peptides wherein said combination of peptides comprises ligands capable of binding to at least 70%, 80%, 90%, 92%, 94%, 95%, 96%, 97%, 98%, 99% or 100% of the HLA class I molecules that are encoded by HLA alleles predominant in the population of human subjects to be treated. Preferred HLA class I ligands in peptides according to the invention are ligands capable of binding to: HLA-A0101; HLA-A0201; HLA-A0206; HLA-A0301; HLA-A1101; HLAA2301; HLA-A2402; HLA-A2501; HLA-A2601; HLA-A2902; HLA-A3001; HLAA3002; HLA-A3101; HLA-A3201; HLA-A3303; HLA-A6801; HLA-A6802; HLAA7401; HLA-B0702; HLA-B0801; HLA-B1301; HLA-B1302; HLA-B1402; HLAB1501; HLA-B1502; HLA-B1525; HLA-B1801; HLA-B2702; HLA-B2705; HLAB3501; HLA-B3503; HLA-B3701; HLA-B3801; HLA-B3901; HLA-B4001; HLAB4002; HLA-B4402; HLA-B4403; HLA-B4601; HLA-B4801; HLA-B4901; HLAB5001; HLA-B5101; HLA-B5201; HLA-B5301; HLA-B5501; HLA-B5601; HLAB5701; HLA-B5801 and HLA-B5802. In a preferred embodiment, the medicinal product or (an) immunogenic composition(s) of the invention or used in the method of the invention comprise(s) a combination of peptides wherein said combination of peptides comprises ligands capable of binding to at least 70%, 80%, 90%, 92%, 94%, 95%, 96%, 97%, 98%, 99% or 100% of the HLA class I and ligands capable of binding to at least 20%, 30%, 40%, 42%, 44%, 45%, 46%, 47%, 48%, 49% or 50% of the HLA class II molecules that are encoded by HLA alleles predominant in the population of human subjects to be treated. In a preferred embodiment, a peptide used in the invention comprises a CTL ligand that shows binding affinity, preferably at least intermediate binding affinity, more preferably high binding affinity to an HLA class I molecules that is encoded by an HLA allele predominant in the population of human subjects to be treated. Preferably, a peptide used in the invention comprises a CTL ligand that shows binding affinity, preferably at least intermediate binding affinity, more preferably high binding affinity to at least one HLA class I molecule of the group of HLA class I molecules consisting of: HLA-A0101; HLA-A0201; HLA-A0206; HLA-A0301; HLA-A1101; HLA-A2301; HLA-A2402; HLA-A2501; HLA-A2601; HLA-A2902; HLA-A3001; HLA-A3002; HLA-A3101; HLA-A3201; HLA-A3303; HLA-A6801; HLA-A6802; HLA-A7401; HLA-B0702; HLA-B0801; HLA-B1301; HLA-B1302; HLA-B1402; HLA-B1501; HLA-B1502; HLA-B1525; HLA-B1801; HLA-B2702; HLA-B2705; HLA-B3501; HLA-B3503; HLA-B3701; HLA-B3801; HLA-B3901; HLA-B4001; HLA-B4002; HLA-B4402; HLA-B4403; HLA-B4601; HLA-B4801; HLA-B4901; HLA-B5001; HLA-B5101; HLA-B5201; HLA-B5301; HLA-B5501; HLA-B5601; HLA-B5701; HLA-B5801 and HLA-B5802.

In a preferred embodiment, an immunogenic peptide of the invention comprises a CTL ligand as described above and a T helper ligand that shows binding affinity, preferably at least intermediate binding affinity, more preferably high binding affinity to an HLA class II molecules that is encoded by an HLA allele predominant in the population of human subjects to be treated.

The immunogenic peptide of the invention may not or may comprise a non-naturally occurring sequence as a result of comprising additional amino acids not originating from the protein or antigen and/or as a result of comprising a modified amino acid and/or a non-naturally occurring amino acid and/or a covalently linked functional group such as a fluorinated group, a fluorocarbon group, a human toll-like receptor ligand and/or agonist, an oligonucleotide conjugate, PSA, a sugar chains or glycan, a Pam3cys and/or derivative thereof, preferably such as described in WO2013051936A1, CpG oligodeoxynucleotides (CpG-ODNs), cyclic dinucleotides (CDNs), a DC pulse cassette, a tetanus toxin derived peptide or a human HMGB1 derived peptide.

### Immunogenic Compositions and Medicinal Products

In a further aspect, the invention relates to an immunogenic composition comprising an immunogenic peptide of the invention as described herein.

In one embodiment, the immunogenic composition comprises an immunogenic peptide of the invention as described herein and a pharmaceutically acceptable carrier. Pharmaceutically-acceptable carriers are well-known in the art.

In one embodiment, the immunogenic peptide in the immunogenic composition of the invention is for more than 85%, for example more than 90%, such as more than 93%, for example more than 95% in the desired form, i.e. the form wherein the disulfide bond is not an intramolecular disulfide bond between two cysteine residues within the same immunogenic peptide molecule and wherein said disulfide bond is also not an intermolecular disulfide bond connecting two peptide molecules. Herein, “%” refers to a percentage of the molecules present in the composition.

Thus, in one embodiment, the immunogenic composition comprises more than 85%, for example more than 90%, such as more than 93%, for example more than 95% of the peptide molecules in a monomeric form lacking intramolecular disulfide bonds.

In one embodiment, the immunogenic composition comprises an immunogenic peptide of the invention and a pharmaceutically-acceptable carrier, wherein said immunogenic peptide is present at a concentration of at least 10 microgram/mL, such as at least 20 microgram/mL, such as at least 30, at least 40 or at least 50 microgram/mL. In another embodiment, the immunogenic composition comprises an immunogenic peptide of the invention and a pharmaceutically-acceptable carrier, wherein said immunogenic peptide is present at a concentration of at most 1 mg/ml.

In one embodiment of the immunogenic composition, the immunogenic peptide is present in an oxidizing environment, for example dissolved in an oxidizing solvent, such as 20% v/v DMSO in water.

In one embodiment, the immunogenic composition comprises at least one further immunogenic peptide, such as a further immunogenic peptide as defined herein (i.e. an immunogenic peptide of 20 to 45 amino acid residues in length comprising one or more cysteine residues, wherein said one or more cysteine residues is present in a modified form that prevents the formation of intermolecular and/or intramolecular disulfide bonds). Thus, the immunogenic composition may contain two or more immunogenic peptides, such as 2, 3, 4, 5, 6, 7, 8, 9, 10 or more immunogenic peptides, for example 2, 3, 4, 5, 6, 7, 8, 9, 10 or more immunogenic peptides of the invention as defined herein. In one embodiment, all immunogenic peptides present in the immunogenic composition of the invention are for more than 85%, for example more than 90%, such as more than 93%, for example more than 95% in the desired form, i.e. the form wherein the disulfide bond is not an intramolecular disulfide bond between two cysteine residues within the same immunogenic peptide molecule and wherein said disulfide bond is also not an intermolecular disulfide bond connecting two peptide molecules. Thus, in one embodiment, the immunogenic composition comprises more than 85%, for example more than 90%, such as more than 93%, for example more than 95% of the peptide molecules in a monomeric form lacking intramolecular disulfide bonds.

In one embodiment, the immunogenic composition comprises:
(i) two or more peptides comprising or consisting of a sequence selected from the group of sequences set forth in SEQ ID NO: 1-6 sequences, or
(ii) two or more peptides comprising or consisting of a sequence selected from the group of sequences set forth in SEQ ID NO: 7-18, or
(iii) two or more peptides comprising or consisting of a sequence selected from the group of sequences set forth in SEQ ID NO: 38, 39, 40 and 44, wherein all cysteines are cysteinylated, i.e. present in the form of a cystine.

In one embodiment, the immunogenic composition comprises one, two, three or all four peptides having the sequence set forth in SEQ ID NO: 38, 39, 40 and 44, wherein all cysteines are cysteinylated, i.e. present in the form of a cystine.

In one embodiment, the immunogenic composition comprises one, two, three or all four peptides having the sequences set forth in SEQ ID NO: 38, 39, 40 and 44, wherein all cysteines are cysteinylated, i.e. present in the form of a cystine, and further comprises one, two, three or four peptides having the sequence set forth in SEQ ID NO: 37, 41, 42 and 43.

In one embodiment, the immunogenic composition:

(i) does not comprise peptides comprising, or consisting of, one of the sequences of the group consisting of SEQ ID NO: 45, SEQ ID NO: 46, SEQ ID NO: 47, in SEQ ID NO:48, SEQ ID NO:49, SEQ ID NO:50 and SEQ ID NO:51 (WO2008118017), or

(ii) does not comprise peptides comprising or consisting of any of the sequences of the group consisting of SEQ ID NO: 45, SEQ ID NO: 46, SEQ ID NO: 47, in SEQ ID NO: 48, SEQ ID NO: 49, SEQ ID NO: 50 and SEQ ID NO: 51.

In one embodiment, the immunogenic composition does not comprise peptides comprising or consisting of any one of the sequence set forth in Table 2, Table 3A or Table 3B, Table 4 or Table 6 of WO2008118017, other than peptides comprising or consisting of the sequences set forth in SEQ ID NO: 37-44 of the present disclosure.

In one embodiment, the immunogenic composition comprises administration of peptides comprising or consisting of the sequences set forth in SEQ ID NO: 37-44, but does not comprise further peptides comprising or consisting of any one of the sequence set forth in Table 2, Table 3A or Table 3B, Table 4 or Table 6 of WO2008118017.

Immunogenic compositions of the invention are preferably for, and therefore formulated to be suitable for, administration to a human subject. The administration may be parenteral, e.g. intravenous, subcutaneous, intramuscular, intradermal, intracutaneous and/or intratumoral administration, i.e. by injection. In a preferred embodiment, the composition is for intradermal administration,

The immunogenic compositions are preferably chemically stable, i.e. the peptides in the composition do not chemically degrade or decompose. Thus, preferably, the amount of un-degraded, un-decomposed and/or unreacted peptides within the solution and/or composition is at least 90%, 91%, 92%, 93%, 94%, 95%, 96%, 97%, 98%, 99% or even 100% by weight as compared to its original, after storage of the solution or liquid composition for at least about 0.5, 1, 1.5, 2 or at least 3 hours at room temperature. Chemical stability can be assessed using any suitable technique known in the art, for instance using UPLC/MS as exemplified herein. When using UPLC/MS, a solution/composition is defined as chemically stable if the total % area of peaks that do not represent the desired peptide product in the UV-chromatogram after storage of at least about 0.5, 1, 1.5, 2 or at least 3 hours at room temperature is at most 20%, 15%, 10%, 9%, 8%, 7%, 6%, 5%, 4%, 3%, 2%, 1%, or 0% as compared to its original.

The immunogenic compositions are preferably also physically stable, i.e. the peptides in the composition do not precipitate or re-disperse. Physical stability can be assessed using any suitable technique known in the art, for instance by visual inspection or by particle distribution using a Malvern Mastersizer as exemplified herein, wherein average particle size is expressed in D (0.5). When using Malvern Mastersizer for assessing physical stability as exemplified herein, a solution/composition is defined as physically stable if the average D (0.5) after storage of at least about 0.5, 1, 1.5, 2 or at least 3 hours at room temperature is increased at most 50%, 40%, 30%, 20%, 10% or 5% as compared to its original (i.e. the freshly prepared solution directly after preparation). Preferably, a solution/composition is defined as physically stable if the average D (0.5) after storage of 3 hours at room temperature is increased at most 50%, 40%, 30%, 20%, 10% or 5%, preferably at most 20%, as compared to its original.

In one embodiment, the immunogenic composition comprises or consists of a mixture of dry or lyophilized peptides that are to be administered together.

In a further aspect, the invention relates to a medicinal product comprising one or more immunogenic peptides of the invention as described herein. A medicinal product may be a single immunogenic composition or comprise more than one immunogenic composition, for example two or more immunogenic compositions each comprising one or more immunogenic peptides, suitable for combined administration to a patient, such as a human patient.

In one embodiment, the medicinal product is a kit comprising two or more parts, e.g. two or more vials, wherein a plurality of immunogenic peptides of the invention are distributed over said two or more parts, e.g. distributed over two or more vials. For example, the peptides may be distributed over two or more immunogenic compositions. In such an embodiment, the compositions may be mixed before administration of the medicinal product to the patient or the compositions may be administered separately. In one embodiment, the medicinal product comprises two or more compositions comprising dried or lyophilized peptides and the medicinal product further comprises a reconstitution solution and optionally an adjuvant, wherein the adjuvant may be comprised within the reconstitution solution or be provided in a further separate vial.

In one embodiment, the medicinal product comprises:
two or more peptides comprising or consisting of a sequence selected from the group of sequences set forth in SEQ ID NO: 1-6, or
two or more peptides comprising or consisting of a sequence selected from the group of sequences set forth in SEQ ID NO: 7-18,
two or more peptides comprising or consisting of a sequence selected from the group of sequences set forth in SEQ ID NO: 38, 39, 40 and 44, wherein all cysteines are cysteinylated, i.e. present in the form of a cystine and wherein the immunogenic peptides optionally are distributed over two or three immunogenic compositions.

### Uses and Medical Treatment

In a further aspect, the invention relates to an immunogenic peptide, an immunogenic composition or a medicinal product according to the invention for use in medicine.

Furthermore, the invention relates to a method of treatment comprising administration of an immunogenic peptide, an immunogenic composition or a medicinal product according to the invention to a subject, such as a human subject, in need thereof.

In one embodiment, the method of treatment or use comprises immunisation of a human subject.

In one embodiment, the method of treatment or use comprises therapeutic immunisation of a human subject for the treatment of cancer.

In one embodiment, the method of treatment or use comprises administration of one, two, three or all four peptides having the sequence set forth in SEQ ID NO: 38, 39, 40 and 44, wherein all cysteines are cysteinylated, i.e. present in the form of a cystine.

In one embodiment, the method of treatment or use comprises administration of all four peptides having the sequences set forth in SEQ ID NO: 38, 39, 40 and 44, wherein all cysteines are cysteinylated, i.e. present in the form of a cystine, and further comprises administration of one, two, three or four peptides having the sequence set forth in SEQ ID NO: 37, 41, 42 and 43.

In one embodiment, the method of treatment or use:
(i) does not comprise administration of peptides comprising, or consisting of, one of the sequences of the group consisting of SEQ ID NO: 45, SEQ ID NO: 46, SEQ ID NO: 47, in SEQ ID NO:48, SEQ ID NO:49, SEQ ID NO:50 and SEQ ID NO: 51 (WO2008118017), or
(ii) does not comprise administration of peptides comprising or consisting of any of the sequences of the group consisting of SEQ ID NO: 45, SEQ ID NO: 46, SEQ ID NO: 47, in SEQ ID NO: 48, SEQ ID NO: 49, SEQ ID NO: 50 and SEQ ID NO: 51.

In one embodiment, the method of treatment or use does not comprise administration of peptides comprising or consisting of any one of the sequence set forth in Table 2, Table 3A or Table 3B, Table 4 or Table 6 of WO2008118017, other than peptides comprising or consisting of the sequences set forth in SEQ ID NO: 37-44 of the present disclosure.

In one embodiment, the method of treatment or use comprises administration of peptides comprising or consisting of the sequences set forth in SEQ ID NO: 37-44, but does not comprise administration of further peptides comprising or consisting of any one of the sequence set forth in Table 2, Table 3A or Table 3B, Table 4 or Table 6 of WO2008118017.

The method or use of the invention wherein PRAME-based SLPS are administered is typically for the treatment or prevention of a PRAME-expressing cancer. In one embodiment, the cancer is selected from the group consisting of: neuroblastoma, lymphoma, papillomas, breast or cervical carcinomas, acute and chronic leukemias, medulloblastoma, non-small cell lung carcinoma, head and neck cancer, renal carcinoma, pancreatic carcinoma, prostate cancer, small cell lung cancer, multiple myeloma, melanoma, uveal melanoma, sarcomas and hematological malignancies like chronic myeloid leukemia and acute myeloid leukemia.

In one embodiment, the method of treatment or use comprises prophylactic immunisation of a human subject against a pathogen.

In one embodiment, the method of treatment or use does not comprise induction of tolerance in the human subject.

In one embodiment, the method of treatment or use comprises prophylactic immunisation of a human subject against a viral pathogen, such as hepatitis B virus or human papillomavirus.

In one embodiment, the method of treatment or use comprises administration to a human subject of two or more immunogenic peptides of the invention, such as 3 or more, such as 4 or more, e.g. 5 or more, such as 6 or more, e.g. 7 or more, such as 8 or more, e.g. 9 or more, such as 10 or more, e.g. 11 or more, such as 12 or more, e.g. 13, 14, 15, 16, 17, 18, 19, or 20 or more of the immunogenic peptides of the invention.

In one embodiment, the method of treatment or use comprises intradermal administration of the immunogenic peptide, immunogenic composition or medicinal product of the invention.

In one embodiment, the method of treatment or use comprises in inducing a CD8+ T cell response in a human subject.

If the immunogenic peptides to be used are divided over two or more compositions, these compositions may be mixed prior to administration and thus be co-administered, or they may be administered separately. Typically, all compositions, will be administered to the subject in a time interval of 24 hours, preferably within 4, 2 or 1 hour. If two or more compositions are administered, the administration may be at the same injection site, e.g. in the same limb, or at two or more different injection sites.

In the course of the treatment, the administration of the composition(s) may be carried out once or alternatively may be repeated (boosted) subsequently, such as, but not limited to, twice or three times. In one embodiment, a boost immunization is given after less than 28 days after the first immunization, such as less than 21 days, e.g. after between 5 and 20 days, such as after 7, 10 or 14 days. In one embodiment, the immunogenic composition used for the boost immunization has the same composition as the initially administered composition.

In another embodiment, the immunization is a single-dose vaccination, i.e. is not repeated within 6 months.

### Adjuvants

In one embodiment, an immunogenic composition or medicinal product of the invention further comprises an adjuvant or the method of treatment or use further includes administration of an adjuvant.

The term “adjuvant” is used herein to refer to substances that have immune-potentiating effects and are co-administered, or added to, or co-formulated with an antigenic agent in order to enhance, induce, elicit, and/or modulate the immunological response against the antigenic agent when administered to a subject. In one embodiment, the adjuvant is physically linked, such as covalently linked, to the peptide(s) to be reconstituted.

In one embodiment, the adjuvant is an emulsifying adjuvant. In one embodiment, the adjuvant is an oil-based adjuvant. Oil-based adjuvants can be used to form emulsions (e.g. water-in-oil or oil-in-water emulsions) and are appreciated in the art to enhance and direct the immune response. Preferably the oil-based adjuvant is a mineral oil-based adjuvant. Non-limiting examples of oil-based adjuvants are bio-based oil adjuvants (based on vegetable oil/fish oil, etc.), squalene-based adjuvant (e.g. MF59), Syntex Adjuvant Formulation (SAF; Lidgate, Deborah M, Preparation of the Syntex Adjuvant Formulation (SAF, SAF-m, and SAF-1), In: Vaccine Adjuvants, Volume 42 of the series Methods in Molecular Medicine™ p229-237, ISSN1543-1894), Freund's Complete Adjuvant (FCA), Freund's Incomplete Adjuvant (FIA), adjuvants based on peanut oil (e.g. Adjuvant 65), Lipovant (Byars, N. E., Allison, A. C., 1990. Immunologic adjuvants: general properties, advantages, and limitations. In: Zola, H. (Ed.), Laboratory Methods in Immunology. p39-51), ASO4 (A. Tagliabue, R. Rappuoli Vaccine adjuvants: the dream becomes real Hum. Vaccine, 4 (5), 2008, p347-349), Montanide adjuvants, which are based on purified squalene and squalene emulsified with highly purified mannide mono-oleate (e.g. Montanide ISA 25 VG, 28 VG, 35 VG, 50 V, 50 V2, 51 VG, 61 VG, 70 VG, 70 M VG, 71 VG, 720 VG, 760 VG, 763 A VG, 775 VG, 780 VG, 201 VG, 206 VG, 207 VG). More preferably, the oil-based adjuvant is Montanide ISA 51VG (Seppic), which is a mixture of Drakeol VR and mannide monooleate.

Other suitable adjuvants are adjuvants that are known to act via the Toll-like receptors and/or via a RIG-I (Retinoic acid-Inducible Gene-1) protein and/or via an endothelin receptor. Immune modifying compounds that are capable of activation of the innate immune system can be activated particularly well via Toll like receptors (TLRs), including TLRs 1-10. Compounds capable of activating TLR receptors and modifications and derivatives thereof are well documented in the art. TLR1 may be activated by bacterial lipoproteins and acetylated forms thereof, TLR2 may in addition be activated by Gram positive bacterial glycolipids, LPS, LPA, LTA, fimbriae, outer membrane proteins, heat shock proteins from bacteria or from the host, and Mycobacterial lipoarabinomannans. TLR3 may be activated by dsRNA, in particular of viral origin, or by the chemical compound poly(I:C). TLR4 may be activated by Gram negative LPS, LTA, Heat shock proteins from the host or from bacterial origin, viral coat or envelope proteins, taxol or derivatives thereof, hyaluronan containing oligosaccharides and fibronectins. TLR5 may be activated with bacterial flagellae or flagellin. TLR6 may be activated by mycobacterial lipoproteins and group B Streptococcus heat labile soluble factor (GBS-F) or Staphylococcus modulins. TLR7 may be activated by imidazoquinolines, such as imiquimod, resiquimod and derivatives imiquimod or resiquimod (e.g. 3M-052). TLR9 may be activated by unmethylated CpG DNA or chromatin-IgG complexes. Particularly preferred adjuvants comprise, but are not limited to, synthetically produced compounds comprising dsRNA, poly(I:C), poly I:CLC, unmethylated CpG DNA which trigger TLR3 and TLR9 receptors, IC31, a TLR 9 agonist, IMSAVAC, a TLR4 agonist, a water-in-oil emulsion comprising a mineral oil and a surfactant from the mannide monooleate family (e.g. Montanide ISA-51, Montanide ISA 720 an adjuvant produced by Seppic, France). RIG-I protein is known to be activated by ds-RNA just like TLR3 (Kato et al, (2005) Immunity, 1:19-28).

A further particularly preferred TLR ligand is a Pam3cys and/or derivative thereof, preferably a Pam3cys lipopeptide or variant or derivative thereof, preferably such as described in WO2013051936A1 (incorporated herein by reference), more preferably U-Pam12 (SEQ ID NO:34) or U-Pam14 (SEQ ID NO:35) a.k.a. AMPLIVANT®.

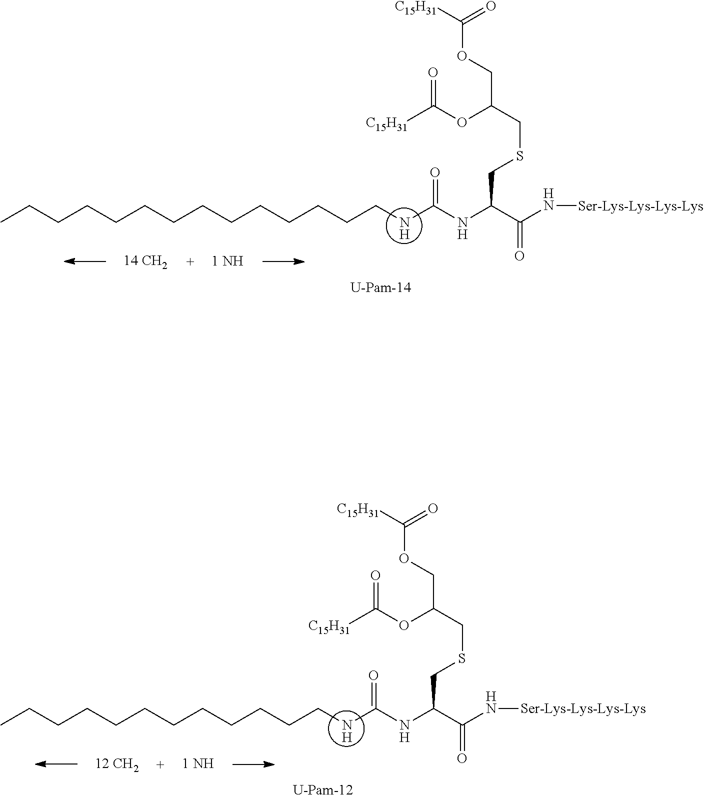

Pam3cys and/or derivatives thereof may optionally be covalently linked to the peptide antigen(s).

Further preferred adjuvants are Cyclic dinucleotides (CDNs), Muramyl dipeptide (MDP) and poly-ICLC. In a preferred embodiment, the adjuvants of the invention are non-naturally occurring adjuvants such as the Pam3cys lipopeptide derivative as described in WO2013051936A1, Poly-ICLC, imidazoquinoline such as imiquimod, resiquimod or derivatives thereof, CpG oligodeoxynucleotides (CpG-ODNs), such as class A-ODN (or K-type), class B-ODN (or D-type), class C-ODN as described in Sheiermann and Klinman, 2014 Vaccine 32 (48): 6377-6389, more preferably class B-ODN (such as CpG7909 or 1018ISS) or class C-ODN (such as DV-281), having a non-naturally occurring sequence, and peptide-based adjuvants, such as muramyl dipeptide (MDP) or tetanus toxoid peptide, comprising non-naturally occurring amino acids.

Further preferred are adjuvants selected from the group consisting of: aluminum salts, Amplivax, AS 15, BCG, CP-870,893, CyaA, dSLIM, GM-CSF, IC30, IC31, ImuFact EV1P321, IS Patch, ISS, ISCOMATRIX, JuvImmune, LipoVac, MF59, monophosphoryl lipid A, Montanide IMS 1312, OK-432, OM-174, OM-197-MP-EC, ONTAK, PepTel®, vector system, PLGA microparticles, SRL172, Pam3Cys-GDPKHPKSF, YF-17D, VEGF trap, R848, beta-glucan, Aquila's QS21 stimulon, vadimezan, AsA404 (DMXAA), STING (stimulator of IFN genes) agonist (e.g. c-di-GMP VacciGrade™), PCI, NKT (natural killer T cell) agonist (e.g. alpha-galactosylceramide or alpha-GalCer, RNAdjuvant® (Curevac), retinoic acid inducible protein I ligands (e.g. 3pRNA or 5′-triphosphate RNA).

In a preferred embodiment, the method of the invention further comprises administration of an adjuvant, wherein said adjuvant preferably is:
a water-in-oil emulsion comprising a mineral oil and a surfactant from the mannide monooleate family, optionally combined with a TLR9 agonist, or
a TLR2 ligand.

### Dosages

Preferably, the medicinal product or immunogenic composition of the invention comprises or consists of an amount of immunogenic peptides that constitutes a pharmaceutical dose. A pharmaceutical dose is defined herein as the amount of active ingredients (i.e. the total amounts of immunogenic peptides according to the invention in a peptide-based medicinal product) that is applied to a subject at a given time point. A pharmaceutical dose may be applied to a subject in a single volume, i.e. a single shot, or may be applied in 2, 3, 4, 5 or more separate volumes that are applied preferably at different locations of the body, for instance in the right and the left limb. Reasons for applying a single pharmaceutical dose in separate volumes may be multiples, such as avoid negative side effects, avoiding antigenic competition and/or composition analytics considerations.

A pharmaceutical dose may be an effective amount or part of an effective amount. An “effective amount” is to be understood herein as an amount or dose of active ingredients required to prevent and/or reduce the symptoms of a disease relative to an untreated patient. The effective amount of active compound(s) used to practice the present invention for preventive and/or therapeutic treatment of a disease or condition varies depending upon the manner of administration, the age, body weight, and general health of the subject. Ultimately, the attending physician or veterinarian will decide the appropriate amount and dosage regimen. Such amount is referred to as an “effective” amount. This effective amount may also be the amount that is able to induce an effective cellular T cell response or B cell response in the subject to be treated.

Preferably, pharmaceutical dose, or total amount of immunogenic peptides applied to a subject at a given time point, either in a single or in multiple injections at a certain time point, comprises an amount of peptides in the range from 0.1 μg to 20 mg, such as about 0.1 μg, 0.5 μg, 1 μg, 5 μg, 10 μg, 15 μg, 20 μg, 30 μg, 40 ug, 50 μg, 60 μg, 70 μg, 80 μg, 90 μg, 100 μg, 150 μg, 200 μg, 250 μg, 300 μg, 350 μg, 400 μg, 450 μg, 500 μg, 650 μg, 700 μg, 750 μg, 800 μg, 850 μg, 900 μg, 1 mg, 1.1 mg, 1.2 mg, 1.3 mg, 1.4 mg, 1.5 mg, 1.6 mg, 1.7 mg, 1.8 mg, 1.9 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 15 mg or about 20 mg or any value in between. Preferred ranges of pharmaceutical doses are from 0.1 μg to 20 mg, 1 ug to 10 mg, 5 μg to 1 mg, 10 μg to 5 mg, 0.5 mg to 2 mg, 0.5 mg to 10 mg or 1 mg to 5 mg or 2 to 4 mg.

In one embodiment, the medicinal product or immunogenic composition of the invention is administered in a dose of between 1 μg and 300 μg, e.g. between 50 μg and 150 μg, such as approximately 100 μg of each peptide.

A single injection volume (i.e. volume applied on one location at a certain time point), comprising a total pharmaceutical dose, may be between 50 UL and 2 mL, or between 50 UL and 1 mL. The single injection volume may be 50 μL, 100 UL, 200 μL, 300 μL, 400 μL, 500 L, 600 μL, 700 UL, 800 UL, 900 μL, 1 mL, 1.1 mL, 1.2 mL, 1.3 mL, 1.4 mL, 1.5 mL, 1.6 mL, 1.7 mL, 1.8 mL, 1.9 mL, 2 mL, 3 mL or any value in between.

### Methods for Producing Immunogenic Peptides or Immunogenic Compositions

In a further aspect, the invention relates to a method for producing an immunogenic peptide according to the invention, comprising synthesizing the immunogenic peptide chemically. In one embodiment, the peptide is synthesized using solid phase peptide synthesis, for example using the method described in the Examples herein.

In one embodiment of the method for producing an immunogenic peptide according to the invention, the method comprises:
(i) synthesis of the peptide on a solid phase, such as a resin,
(ii) rendering the thiol group of cysteine moiety/moieties in the peptide more reactive, for example by reaction with DTNP (2,2′-dithiobis(5-nitropyridine)), and
(iii) formation of one or more cystine through coupling with free cysteine.

In one embodiment, step (ii) is performed while the peptide is being released from the solid phase.

In another embodiment, step (ii) is performed after the peptide has been released from the solid phase, said release step optionally including a wash step.

Step (ii) may be followed by a (further) wash or purification step.

In a further aspect, the invention relates to a method for producing a stable immunogenic composition comprising one or more immunogenic peptides according to the invention, said method comprising:
(i) synthesis of the peptide on a solid phase, such as a resin,
(ii) rendering the thiol group of cysteine moiety/moieties in the peptide more reactive, for example by reaction with DTNP (2,2′-dithiobis(5-nitropyridine)), and
(iii) formation of one or more cystine through coupling with free cysteine.

In a further aspect, the invention relates to a method for producing a stable immunogenic composition suitable for inducing a therapeutic or prophylactic response in a human patient, said composition comprising one or more immunogenic peptides according to the invention, said method comprising:
(i) synthesis of the peptide on a solid phase, such as a resin,
(ii) rendering the thiol group of cysteine moiety/moieties in the peptide more reactive, for example by reaction with DTNP (2,2′-dithiobis(5-nitropyridine)), and
(iii) formation of one or more cystine through coupling with free cysteine.

In a further aspect, the invention relates to a method for producing an immunogenic composition according to the invention, comprising dissolving one or more immunogenic peptides according to the invention in a solvent. Immunogenic compositions and medicinal products of the invention may be prepared by any suitable method. In some embodiments, the immunogenic composition(s) are prepared from dried, preferably lyophilized, immunogenic peptides. For example, the composition may be prepared by a method comprising the following steps:
a) providing a vial comprising dried, preferably lyophilized, peptides;
b) thawing the peptides, preferably for about 5-30 min;
c) adding a reconstitution solution to the vial comprising the peptides, preferably without swirling the vial;
d) allowing to admix, preferably for about 0.5-5 minutes; and
e) swirling until a clear solution is obtained, preferably for about 1-3 minutes. Preferably, steps b) to e) are performed at room temperature.

Preferably, said vial comprises peptides in an amount for injection as a single volume in a method of treatment as defined herein, i.e. a single pharmaceutical dosage unit, or part thereof in case of multiple injections at difference locations of the subject's body at substantially the same time point.

In one embodiment, the reconstitution solution of step c) comprises or consists of DMSO and/or water-for-injection.

Dried peptides may be peptides free of further constituents but may also comprise buffer components or excipients, such as trifluoroacetic acid (TFA), salts such as sodium, potassium or phosphate salts (e.g. NaCl, KCl and NaPO₄). The amount of further constituents is preferably less than 30%, more preferably less than 25%, of the total weight of the dry peptides to be reconstituted. Dried peptides to be reconstituted may be in a physical dried state as can be obtained by processes such as, but not limited to, rotor evaporation, lyophilization (freeze drying) and spray drying.

All patent and literature references cited in the present specification are hereby incorporated by reference in their entirety.

### EXAMPLES

### Example 1: General Method for Synthesis of Cystine-Containing Peptides

All reagents and solvents for solid phase peptide synthesis were purchased from Merck, Sigma Aldrich, Actu-All, Bachem and Biosolve, GL Biochem and used as received.

### Solid Phase Peptide Synthesis (SPPS)

Peptides synthesis was performed on a Tetras peptide synthesizer (Advanced ChemTech) by solid phase Fmoc/′Bu chemistry according to established methods. In general, the peptide synthesis was carried out using pre-loaded Wang-, HMPB ChemMatrix®, 2-chlorotrityl, or 4-(1′,1′-dimethyl-1′-hydroxypropyl) phenoxyacetyl-alanyl-aminomethyl resin. Reactions were typically carried out on a 30 to 60 mmol scale. The peptides were synthesized by single, double or triple coupling cycles or a combination of single, double and triple coupling cycles.

The initial swell procedure
1) Swelling of the resin: 2 cycles with NMP
2) NMP wash
3) iPrOH wash

A single coupling cycle was performed by the following consecutive steps:
1) Deprotection of the Fmoc-group: 3 cycles with piperidine in N-methyl-2-pyrrolidone (NMP).
2) NMP wash
3) iPrOH wash
4) Coupling of the appropriate amino acid. After addition of the Fmoc-amino acid in NMP and the coupling reagent (3-(diethoxy-phosphoryloxy)-1,2,3-benzo[d] triazin-4 (3H)-one (DEPBT), 2-(1H-benzotriazole-1-yl)-1,1,3,3-tetramethylaminium hexafluorophosphate (HBTU), 2-(1H-benzotriazol-1-yl)-N,N,N′,N′-tetramethylaminium tetrafluoroborate (TBTU), benzotriazole-1-yl-oxy-tris-pyrrolidino-phosphonium hexafluorophosphate (PyBOP), 7-aza-benzotriazol-1-yloxy-tripyrrolidinophosphonium hexafluorophosphate (PyAOP), Ethyl cyano (hydroxyimino) acetato-O2-tri-(1-pyrrolidinyl)-phosphonium hexafluorophosphate (PyOxim), 1-[bis(dimethylamino)methylene]-1H-1,2,3-triazolo[4,5-b]pyridinium 3-oxid hexafluorophosphate (HATU), 1-[1-(Cyano-2-ethoxy-2-oxoethylideneaminooxy)-dimethylamino-morpholino]-uronium hexafluorophosphate (COMU), 2-(1-Oxy-pyridin-2-yl)-1,1,3,3-tetramethylisothiouronium tetrafluoroborate (TOTT),O-(1H-6-chlorobenzotriazole-1-yl)-1,1,3,3-tetramethyluronium hexafluorophosphate (HCTU), N,N′-diisopropylcarbodiimide (DIC) or ethyl 2-cyano-2-(hydroximino) acetate (Oxyma Pure®)) in NMP, the reaction mixture was shaken for 1 min. Optionally a base such as N-methylmorpholine (NMM) or N,N-diisopropylethylamine (DIPEA) was added. The reaction mixture was shaken for at least 30 min.
5) NMP wash
6) Capping by acetic anhydride or benzoyl chloride in the presence of a base (NMM or pyridine).
7) NMP wash
8) iPrOH wash

A double coupling cycle was performed by the following consecutive steps:
1) Deprotection of the Fmoc-group: 3 cycles with piperidine in NMP.
2) NMP wash
3) iPrOH wash
4) First coupling cycle: After addition of the Fmoc-amino acid in NMP and the coupling reagent (DEPBT, HBTU, TBTU, PyBOP, PyAOP, PyOxim HATU, COMU, TOTT, HCTU, DIC or Oxyma Pure®) in NMP, the reaction mixture was shaken for 1 min. Optionally a base (NMM or DIPEA) was added. The reaction mixture was shaken for at least 15 min.
5) Purge of the reaction vessel.
6) Second coupling cycle: After addition of the Fmoc-amino acid in NMP and the coupling reagent (DEPBT, HBTU, TBTU, PyBOP, PyAOP, PyOxim, HATU, COMU, TOTT, HCTU, DIC or Oxyma Pure®)) in NMP, the reaction mixture was shaken for 1 min. Optionally a base (NMM or DIPEA) was added. The reaction mixture was shaken for at least 15 min.
7) NMP wash
8) Capping by acetic anhydride or benzoyl chloride in the presence of a base (NMM or pyridine).
9) NMP wash
10) iPrOH wash

A triple coupling cycle was performed by the following consecutive steps:
1) Deprotection of the Fmoc-group: 3 cycles with piperidine in NMP.
2) NMP wash
3) iPrOH wash
4) First coupling cycle: After addition of the Fmoc-amino acid in NMP and the coupling reagent (DEPBT, HBTU, TBTU, PyBOP, PyAOP, PyOxim HATU, COMU, TOTT, HCTU, DIC or Oxyma Pure®) in NMP, the reaction mixture was shaken for 1 min. Optionally a base (NMM or DIPEA) was added. The reaction mixture was shaken for at least 15 min.
5) Purge of the reaction vessel.
6) Second coupling cycle: After addition of the Fmoc-amino acid in NMP and the coupling reagent (DEPBT, HBTU, TBTU, PyBOP, PyAOP, PyOxim, HATU, COMU, TOTT, HCTU, DIC or Oxyma Pure®)) in NMP, the reaction mixture was shaken for 1 min. Optionally a base (NMM or DIPEA) was added. The reaction mixture was shaken for at least 15 min.
7) Purge of the reaction vessel.
8) Third coupling cycle: After addition of the Fmoc-amino acid in NMP and the coupling reagent (DEPBT, HBTU, TBTU, PyBOP, PyAOP, PyOxim, HATU, COMU, TOTT, HCTU, DIC or Oxyma Pure®)) in NMP, the reaction mixture was shaken for 1 min. Optionally a base (NMM or DIPEA) was added. The reaction mixture was shaken for at least 15 min.
9) NMP wash
10) Capping by acetic anhydride or benzoyl chloride in the presence of a base (NMM or pyridine).
11) NMP wash
12) iPrOH wash
After the last coupling cycle, the following final Fmoc-deprotection step and wash steps were performed:
1) Deprotection of the Fmoc-group: 3 cycles with piperidine in NMP.
2) NMP wash
3) iPrOH wash
   Cleavage and Purification Procedure with Cysteine Modification During Cleavage

After the final wash steps, the resin was dried and cooled. A cleavage cocktail based on Milli-Q water, thioanisole, and TFA was added to the resin, immediately followed by the addition of 50 equivalents of 2,2′-dithiobis(5-nitropyridine) (DTNP) per cysteine(Trt) residue present in the peptide. The mixture was left standing at room temperature for 90 minutes. Next, the cleavage mixture was filtered and the residue was washed with diethyl ether. The filtrate was collected and centrifuged. The supernatant was removed and fresh diethyl ether was added to the pellet and resuspended by vortexing. After centrifugation, the pellet was isolated and dried under vacuum.

The crude peptide was dissolved in a mixture based on Milli-Q water, MeCN and acetic acid. After centrifugation, the supernatant was isolated. L-cysteine (145 mg, for a SPPS synthesis at a scale of 60 μmol) was added to the supernatant. After 1 h, the reaction mixture was diluted with Milli-Q water and filtered. The peptide was purified by a Waters AutoPurification HPLC/MS system under acidic conditions (ACN, water and TFA) followed by lyophilization overnight to obtain the cystine-containing peptide as a white to off-white powder.

Cleavage and Purification Procedure with Cysteine Modification Off-Resin

After the final wash steps in the peptide synthesizer, the resin was dried and cooled. A cleavage cocktail based on Milli-Q water, ethanethiol, triisopropylsilane, and TFA was added to the resin and mixed for 3 h. Subsequently, cold diethylether was added and the mixture was centrifuged. The supernatant was removed and the pellet was isolated. The following steps were identical as to the above described “cleavage and purification procedure with cysteine modification during cleavage”. Briefly, the obtained filtrate was treated with the cleavage cocktail and DTNP. After 90 minutes, the solution was filtered into diethyl ether. The filtrate was collected, centrifuged and the obtained pellet was washed a second time. Next, the pellet was dissolved in Milli-Q water, MeCN and acetic acid and centrifuged. L-Cysteine was added to the supernatant. After 30 minutes, the reaction mixture was filtered, purified by HPLC/MS system under acidic conditions, followed by lyophilization overnight to obtain the cystine-containing peptide as a white to off-white powder.

### Analysis of the Peptide

The identity and purity of the purified peptides were determined by UPLC-UV-MS on a Waters Acquity UPLC/TQD system using an C18 Waters Acquity BEH130 analytical column (1.7 μm particle size, 2.1×150 mm, flow 0.4 mL/min) with a linear gradient (5% B to 95% B, linear gradient in 10 min). The absorbance was measured at 220 nm.

### Solvent System:

A: 0.05% trifluoroacetic acid (TFA) and 1% ACN in H₂O
B: 0.05% TFA in ACN

The determination confirmed the identity of the synthesized peptides.

### Example 2: General Method for Synthesis of Methionine-Sulfoxide-Containing Peptides

Methionine sulfoxide containing peptides were manufactured by SPPS. The SPPS procedure and analysis of the peptide is described in Example 1, (vide supra). To introduce the methionine sulfoxide in the sequence N-α-Fmoc-L-methionine-DL-sulfoxide was used as building block in the SPPS. After synthesis, the cysteine(s) in cysteine containing peptides were converted to cystine(s) as described in Example 1.

Cleavage and Purification Procedure of Peptides without Introduction of a Cystine

After the final wash steps, the resin was dried, cooled and treated with a trifluoroacetic acid (TFA) based cleavage cocktail. A combination of scavengers such as H₂O, 1,2-ethanedithiol (EDT), triisopropylsilane (TIPS), 3,6-dioxa-1,8-octane-dithiol (DODT), ethanethiol (ET), triethylsilane (TES), phenol, thioanisole, 1-dodecanethiol, 1,4-dithioerythritol (DTE) or dithiothreitol (DTT) were added to the cleavage cocktail. After filtration of the resin, the reaction mixture was shaken at room temperature. Subsequently, the peptide was precipitated in an ether-based solution, centrifuged and the supernatant was removed. The solid precipitate was resuspended in an ether-based solution, centrifuged and the supernatant was removed. The resulting pellet was dissolved in a H₂O based mixture with acetonitrile (ACN) and TFA or with acetic acid and lyophilized overnight. The peptide was purified by a Waters AutoPurification HPLC/MS system under acidic conditions (ACN, water and TFA) followed by lyophilization overnight to obtain the methionine-sulfoxide-containing peptide as a white to off-white powder.

### Example 3: Stability of Cysteine and Cystine-Containing Peptides

To examine the effect on stability of a peptide that contains cysteine(s) (non-oxidized, C) or cystine(s) (oxidized, C (C)) in a formulation matrix (20% v/v DMSO in WFI (water for injection), four non-oxidized (SEQ ID NO: 1, 2, 3 and 4) and their oxidized, cystine-containing, peptides were manufactured and individually examined in 20% v/v DMSO (aq.)

### Method

Each peptide (2.0 mg) was dissolved in 2.0 ml of 0.09% TFA in H₂O: acetonitrile (1:1, v/v). Aliquots of 200 μL were transferred to a 1 mL amber vial and then freeze dried overnight. An aliquot (0.2 mg) was then redissolved in 20% v/v DMSO in WFI (1 mL). After swirling for 2 minutes, a sample was analyzed by UPLC-UV-MS. The remainder of the sample was stored at room temperature and analyzed at different time intervals (1 h, 2 h, 3 h, 4 h). The identity and purity of the purified peptides were then determined by UPLC-UV-MS on a Waters Acquity UPLC/TQD system using an C18 Waters Acquity BEH130 analytical column (1.7 μm particle size, 2.1×150 mm, flow 0.4 mL/min) with a linear gradient (5% B to 95% B, linear gradient in 10 min). The absorbance was measured at 220 nm.

MassLynx v4.1 was used to integrate the individual peaks of the chromatogram in the region of the observed peptide-derived signals. The following ApexTrack peak detection parameters were used for integration: peak-to-peak baseline noise was set at automatic, peak width at 5% height was set at automatic, baseline start threshold of 0.05% and baseline end threshold of 0.05%. The detect shoulder function was turned on. The following response thresholds were set: relative height of 0.20 and relative area of 0.10. After the peak of the desired peptide was identified by MS, the corresponding integral of the peak in the UV chromatogram is reported in Table 2 as percentage of the total integrated peaks, purity (% a/a).

### Solvent System:

A: 0.05% trifluoroacetic acid (TFA) and 1% ACN in H₂O
B: 0.05% TFA in ACN

### Results

Purity of the non-oxidized and oxidized peptide composition over time was evaluated over 4 h. Additional mass adducts, consistent with the formation of intermolecular or intramolecular disulfide bonds, increased over time in all spectra of cysteine-containing peptides compared to cystine-containing peptides. The purity of the non-oxidized peptides set forth in SEQ ID NO: 1, 3 and 4 declined from >90% a/a at to to less than 80% a/a with the lowest stability observed for SEQ ID NO: 1 with a purity of 64% a/a after 4 h. For the cystine-containing peptide analogs, the purity remained >90% a/a after 4 h. Thus, the cystine-containing peptide analogs are significantly more stable than their cysteine-containing counterparts.

TABLE 2
Stability data of cysteine-containing peptides SEQ ID NO: 1,
SEQ ID NO: 3 and SEQ ID NO: 4 and the
cystine analog peptides in 20% v/v DMSO in WFI for 4 h.
The purity is expressed as an area percentage (% a/a) of the
intended peptide as percentage of the total response.

| | Purity (% a/a) | | | | | |
|---|---|---|---|---|---|---|
| | Cysteine (C) containing | | | Cystine(C(C)) containing | | |
| | SEQ | SEQ | SEQ | SEQ | SEQ | SEQ |
| Time | ID | ID | ID | ID | ID | ID |
| (h) | NO: 1 | NO: 3 | NO: 4 | NO: 1 | NO: 3 | NO: 4 |
| 0 | 95.01 | 97.66 | 96.51 | 97.14 | 97.44 | 93.50 |
| 1 | 86.88 | 93.48 | 90.72 | 96.67 | 97.48 | 94.26 |
| 2 | 78.20 | 87.94 | 83.13 | 95.10 | 97.28 | 93.58 |
| 3 | 70.34 | 84.12 | 77.16 | 95.97 | 97.30 | 93.16 |
| 4 | 64.12 | 79.81 | 72.13 | 95.74 | 97.34 | 92.89 |

### Example 4: Stability of Peptide Mixtures

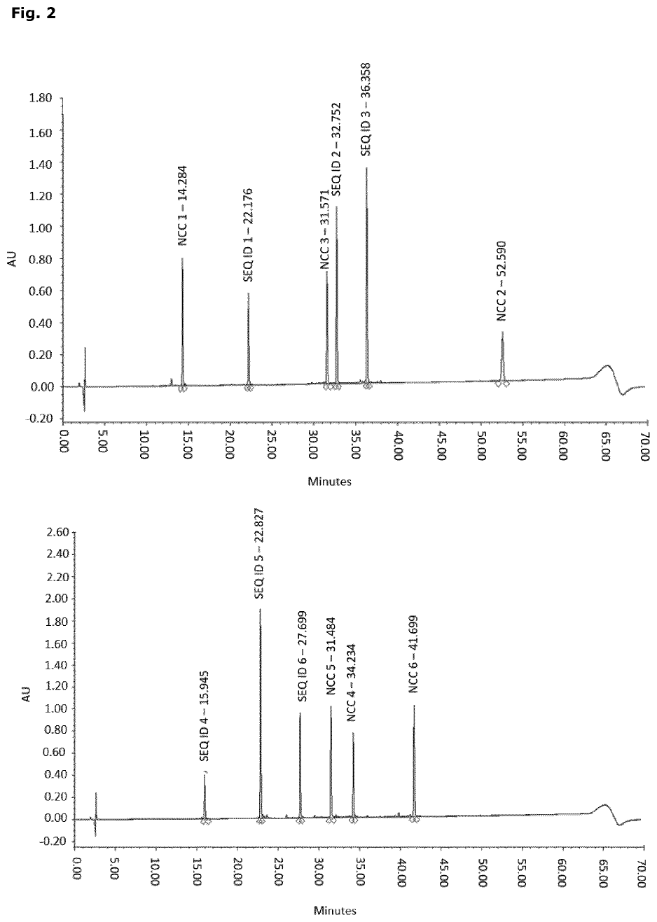

To examine the effect on stability of peptides that contain cystine (oxidized, C (C)) present in a mixture of 6 peptides, two examples of mixtures of both non-cysteine-containing (NCC1-6) and cystine-containing (SEQ ID NO: 1-6) peptides were assessed. Typical chromatograms of the two mixtures are depicted in FIG. 2. Both mixtures were placed on two different storage conditions, ‘long term storage condition’ (i.e. −20±5° C.) and ‘accelerated storage condition’ (i.e. 5±3° C.).

The purity, the level of impurities, and the quantification per peptide of both mixtures are determined by reversed phase rapid UPLC using a mobile phase gradient of ACN/IPA and water with TFA, and UV detection at λ=220 nm. The method quantifies main peaks and impurities in mixtures of peptides. The reporting threshold for the method is 0.10% a/a.

Both mixtures of six peptides were stored under the two conditions described and by using the analytical method as described. The stability was monitored. The quantity of each of the peptides present in the mix (assay values), the purity, and the individual impurities were measured at several timepoints over a period of 3 months. Purity remained stable at around 92-93% a/a and the quantity of each of the peptides did not change either comparing to the initial levels (Tables 3 and 4). Furthermore, focussing on the individual impurities it is shown that no significant degradation has taken place as all levels remain constant and no new impurities have formed. These results confirm that the modified peptides of the invention are also stable in peptide mixtures.

TABLE 3
Overview of the results for a first mixture of 6 peptides
including three with cystine modifications.

| | | | Storage at | | Storage at | | |
|---|---|---|---|---|---|---|---|
| | | | −20 ± 5° C. | | 5 ± 3° C. | | |
| Test | | Initial | T2M | T3M | T1M | T2M | T3M |
| Purity (% a/a) | | 94 | 94 | 95 | 93 | 94 | 93 |
| Total related | | 6.1 | 6.2 | 6.0 | 6.6 | 5.6 | 6.5 |
| substances (% a/a) | | | | | | | |
| Assay | SEQ | 0.24 | 0.24 | 0.24 | 0.23 | 0.24 | 0.23 |
| (net | ID 1 | | | | | | |
| peptide | NCC1 | 0.24 | 0.24 | 0.24 | 0.23 | 0.24 | 0.24 |
| content) | SEQ | 0.24 | 0.25 | 0.25 | 0.24 | 0.25 | 0.25 |
| (mg/ | ID 2 | | | | | | |
| vial) | NCC2 | 0.26 | 0.26 | 0.25 | 0.26 | 0.25 | 0.26 |
| | NCC3 | 0.25 | 0.24 | 0.25 | 0.24 | 0.24 | 0.24 |
| | SEQ | 0.24 | 0.24 | 0.25 | 0.23 | 0.24 | 0.25 |
| | ID 3 | | | | | | |

TABLE 4
Overview of the results for a second mixture of 6 peptides
including three with cystine modifications.

| | | | Storage at | | Storage at | | |
|---|---|---|---|---|---|---|---|
| | | | −20 ± 5° C. | | 5 ± 3° C. | | |
| Test | | Initial | T2M | T3M | T1M | T2M | T3M |
| Purity (% a/a) | | 92 | 93 | 93 | 93 | 93 | 92 |
| Total related | | 7.8 | 7.0 | 7.0 | 7.5 | 7.1 | 7.1 |
| substances (% a/a) | | | | | | | |
| Assay | SEQ | 0.21 | 0.20 | 0.21 | 0.20 | 0.21 | 0.21 |
| (net | ID 4 | | | | | | |
| peptide | NCC4 | 0.21 | 0.20 | 0.21 | 0.20 | 0.21 | 0.21 |
| content) | NCC5 | 0.21 | 0.20 | 0.22 | 0.21 | 0.22 | 0.22 |
| (mg/vial) | NCC6 | 0.22 | 0.22 | 0.23 | 0.22 | 0.23 | 0.23 |
| | SEQ | 0.20 | 0.20 | 0.22 | 0.20 | 0.21 | 0.22 |
| | ID 5 | | | | | | |
| | SEQ | 0.21 | 0.20 | 0.22 | 0.20 | 0.21 | 0.22 |
| | ID 6 | | | | | | |

### Example 5: Ex Vivo Studies with Cells from Human Donors

Using cells from donors that have successfully cleared HBV infection (HBV resolvers) and healthy donors, we studied the ability of the given SLPs to activate memory T cells in these individuals. Peripheral blood mononuclear cells (PBMCs) isolated from buffy coats from HBV resolvers and healthy donors were screened for T cell reactivity to different oxidized versions of the SLPs by IFNy ELISpot. SLPs containing cystines instead of cysteines (SLP-Cys-ox), prepared as described in Example 1, were tested. Furthermore, SLPs containing cystines instead of cysteines and containing methionine sulfoxide instead of methionine (Example 2) (SLP-Cys/Met-ox) were tested.

In short, cryopreserved PBMC from healthy donors and HBV resolvers were thawed and 2*10⁶ cells were incubated with each individual SLP in a concentration of 3 μM in a 24 wells plate or with 0.175 ug/mL of Candida albicans antigen as a positive control. Two days later, Multiscreen plates were be coated with an IFNY coating antibody overnight at 4° C. The next day, the multiscreen plates were washed 4× with PBS and a-specific binding was prevented by blocking the plates with Iscove's Modified Dulbecco's Medium (IMDM) containing 8% Human Serum (HS) at 37° C. for at least one hour. Cells were harvested from the 24 well plates, washed and counted. Around 150,000 cells were added per well of the coated Multiscreen plate and each sample was tested in triplicate. As a positive control, phytohaemagglutinin (PHA, 1 μg/mL), a potent T cell activator, was added to cells that were not stimulated with SLP. The plate was cultured overnight at 37° C. Thereafter, cells were discarded and the plate was washed using PBS/0.05% Tween-20. The IFNy detection antibody was diluted and added to each well and incubated for 2 hours at room temperature. Next, the plate was washed using PBS/0.05% Tween-20 and then incubated with streptavidin-Alkaline Phosphatase (ALP) for 1 hour at room temperature. The plate was washed with PBS/0.05% Tween-20. BCIP/NPT ALP substrate was filtered and added per well for 10-20 minutes at room temperature for staining.

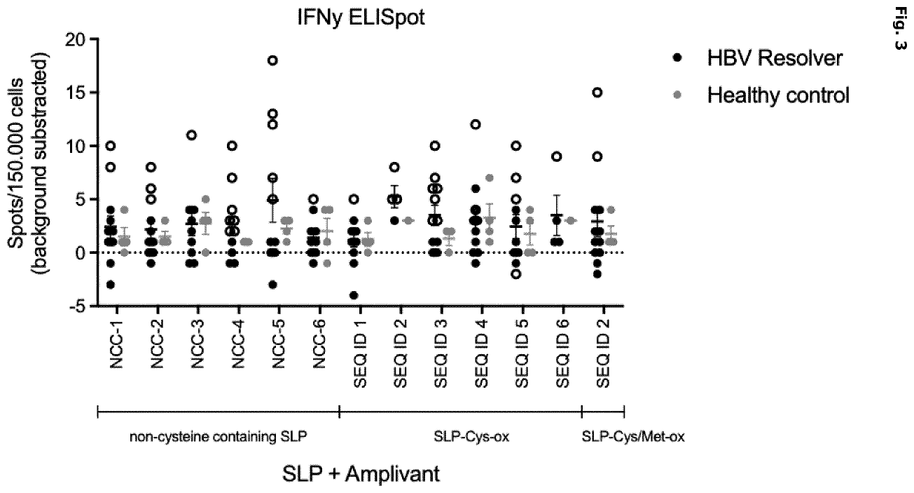

In this study, Amplivant R-isomer was used as adjuvant (in a concentration of 3 μM) in combination with individual SLPs in the 4 day in vitro stimulation for the IFNY ELISpot assay. A-3980-D Albumin peptide (ADCCAKQEPERNECFLQHKDDNPNLPRLVRPEVD (SEQ ID NO:36)) was used as a negative control in the IFNy ELISpot assay. Each SLP was minimally tested on 4 HBV resolvers and 1 healthy donor. As controls, medium only, Albumin peptide control and Candida albicans antigen were included in the culture, PHA was used as a positive control on the ELISpot plate. As shown in FIG. 3, positive responses (indicated by clear symbols) were detected to both SLP-Cys/Met-ox and SLP-Cys-ox. Thus, this study demonstrated SLP-specific T cell responses in blood from HBV resolvers ex vivo to SLP-Cys-ox (cysteine oxidation), and SLP-Cys/Met-ox (cysteine and methionine oxidation).

Overall, this ex vivo study performed with PBMCs obtained from HBV resolvers and healthy donors indicated that the cysteine-oxidized versions of SLPs are immunogenic, as memory T cells were detected using IFNy ELISpot. Addition of Met-oxidation does not affect this effect.

### Example 6: T Cell Induction by PRAME-Based SLPs

The capacity of human-PRAME-based SLPs to activate T cells was studied. The biological activity of the synthesized and purified SLPs was tested using PBMC from healthy donors. Monocytes were isolated using anti-CD14 beads by magnet activated cell sorting (MACS) following the protocol of the supplier (Miltenyi Biotec). In short, PBMCs were isolated by centrifugation over a Ficoll gradient and cryopreserved. To generate dendritic cells (DCs), approximately 50*10 thawed PBMC were used for the isolation of CD14 positive cells. The cells were cultured for three days at 37° C. in 2 ml/well of IMDM 4% human serum (HS) containing 800 U/ml GM-CSF and 500 U/ml IL-4 (Peprotech). After 3 days 1 mL/well of IMDM 4% HS with GM-CSF (2400 U/mL) and IL-4 (1500 U/mL) was added to the monocytes-derived DCs and these adhered cells were cultured for an additional 3 days. On day 6, long peptides distributed over 2 pools were added to monocyte-derived DCs of naive donors at a 13 μM concentration and incubated overnight at 37° C. The next day (day 7) peptide-loaded DCs were harvested, irradiated (1000 rad), washed and mixed in a 1:10 ratio with autologous PBMC in IMDM 8% human serum in the presence of IL-7 (10 ng/ml) and IL-12p70 (100 μg/mL). Conditions in favor of T cell culture, hence from here on referred to as, cultured T cells.

On day 10 new DCs were generated and loaded, after 6 days, with SLPs on day 16 as described above. The next day, these peptide-loaded DCs were harvested, irradiated (1000 rad) and washed. Also the cultured T cells were harvested. Both were counted and mixed in a 1:10 ratio (DC:T) in IMDM 8% human serum in the presence of IL-7 (10 ng/ml) and IL-12p70 (100 μg/mL) for the first restimulation. On the same day, new DCs were generated as described above that were loaded with SLPs on day 23 as described above. The next day, peptide-loaded DCs were harvested, irradiated (1000 rad) and washed. Also the cultured and restimulated T cells were harvested. Both were counted and mixed in a 1:10 ratio (DC:T) in IMDM 8% human serum in the presence of IL-7 (10 ng/ml) and IL-12p70 (100 μg/mL) for the second restimulation.

On this day 24, also test 1 was started in which DCs loaded with individual SLPs were cultured with harvested T cells from restimulation 1. For test 1, DCs and harvested T cells were cultured in a 1:10 ratio for 2 days after which cells were transferred to a coated ELISpot plate (see description below). On day 24, new DCs were generated as described above that were loaded with SLPs on day 30 as described above. The next day, peptide-loaded DCs were harvested, irradiated (1000 rad) and washed. Also the cultured twice restimulated T cells were harvested. Both were counted and mixed in a 1:10 ratio (DC:T) in IMDM 8% human serum in the presence of IL-2 (30 IU/mL) and IL-12p70 (100 μg/mL) for the third restimulation.

On day 31, also test 2 was started in which DCs loaded with individual SLPs were cultured with harvested T cells from restimulation 2. For test 2, DCs and harvested T cells were cultured in a 1:10 ratio for 2 days after which cells were transferred to a coated ELISpot plate (see description below).

The T cell cultures were restimulated three times in 1-week cycles using peptide loaded autologous moDCs. After the 2nd and 3rd restimulation, reactivity towards single SLPs was tested (test 1 and test 2, respectively). Reactivity was determined by measuring IFNy production using ELISpot.

For ELISpot analysis, multiscreen plates were coated with an IFNy coating antibody overnight at 4° C. The next day, the plate was washed 4× with PBS and blocking was done using IMDM 8% HS at 37° C. for at least one hour. Each sample was tested in triplicate. As a positive control, phytoheamagglutinin (PHA, 1 μg/mL) was added to cells that were not stimulated with SLP after thawing. The plate was cultured overnight at 37° C. Thereafter, cells were discarded and the plate was washed using PBS/0.05% Tween-20. The IFNy detection antibody was diluted and added to each well and incubated for 2 hours at room temperature. Next, the plate was washed using PBS/0.05% Tween-20 and then incubated with streptavidin-ALP for 1 hour at room temperature. The plate was washed with PBS/0.05% Tween-20. BCIP/NPT ALP substrate was filtered and added per well for 10-20 minutes at room temperature.

SLPs having the sequences set forth in SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 and SEQ ID NO: 50 were synthesized as described in Example 1. For the cysteine containing SLPs, the cysteinylated SLPs were tested. Each SLP was minimally tested on 7 donors. As controls, medium only, an Albumin-derived peptide control and Candida albicans antigen were included in the culture, PHA was used as a positive control on the ELISpot plate. Positive IFNy responses were detected to 7 of the 10 peptides upon stimulation with 2 peptide pools (Table 5). Next, T cell cultures were generated by stimulating with a pool of peptides consisting only of the three peptides that had not tested positive yet in an additional 6 donors. Upon stimulation of PBMCs with these remaining 3 peptides only, also SEQ ID NO: 39 showed a positive IFNy response (Table 6). Peptides can test positive after the 2nd restimulation (restim2/test1), but not after the 3rd (restim3/test2), as these cultures may then be overgrown by T cells with other specificities. SLPs having the sequences set forth in SEQ ID NO: 45 and SEQ ID NO: 50 did not show convincing responses in any of the 13 donors tested.

In conclusion, 8 of the 10 tested SLPs demonstrated a positive result in these in vitro human T cell cultures, demonstrating their immunogenicity.

TABLE 5
Results of IFNy ELISpot of test 1 and test 2 for the individual SLPs.

| | | SEQ ID | SEQ ID | SEQ ID | SEQ ID | SEQ ID | SEQ ID | SEQ ID | SEQ ID | SEQ ID | SEQ ID |
|---|---|---|---|---|---|---|---|---|---|---|---|
| ELISPOT IFNy | | NO: 42 | NO: 38 | NO: 37 | NO: 50 | NO: 41 | NO: 43 | NO: 45 | NO: 39 | NO: 40 | NO: 44 |
| BC306 | Test 1 | − | − | − | − | − | − | − | − | − | − |
| | Test 2 | − | − | − | − | − | − | − | − | − | − |
| BC312 | Test 1 | − | − | − | − | − | − | − | − | − | − |
| | Test 2 | − | − | − | − | − | − | − | − | − | − |
| BC290 | Test 1 | − | − | + | − | − | nt | nt | nt | nt | nt |
| | Test 2 | − | − | + | − | − | nt | nt | nt | nt | nt |
| BC315 | Test 1 | − | − | − | − | + | nt | nt | nt | nt | nt |
| | Test 2 | − | − | − | − | + | nt | nt | nt | nt | nt |
| BC298 | Test 1 | + | + | − | − | − | − | − | − | − | − |
| | Test 2 | + | + | − | − | − | − | − | − | − | + |
| BC305 | Test 1 | − | − | + | − | − | − | − | − | − | − |
| | Test 2 | − | − | − | − | − | − | − | − | − | − |
| BC321 | Test 1 | + | + | − | − | − | + | − | − | + | + |
| | Test 2 | − | − | − | − | − | − | − | − | + | − |
| | | | POS | POS | NEG | POS | POS | NEG | NEG | POS | POS |

Results are depicted as − (negative) or + (positive).
A response was defined as positive when the average spot count was higher than the average spot count + 2*standard deviation of the irrelevant SLP control.
The last row depicts a summary of the results in the table.
When at least one donor had a positive response, this is denoted as POS.
BCxxx = healthy donor buffy coat codes, nt = not tested.

TABLE 6
Results of IFNy ELISpot of test 1 and test 2 for 3 individual
SLPs that did not test positive in the experiments depicted
in Table 5. These 3 SLPs were tested in an additional 6 donors.
Results are depicted as − (negative) or + (positive).
A response was defined as positive when the average spot count
was higher than the average spot count + 2*standard deviation
of the irrelevant SLP control. The last row depicts a summary
of the results in the table. When at least one donor had a positive
response, this is denoted as POS. BCxxx = healthy donor
buffy coat codes, nt = not tested.

| | | | |
|---|---|---|---|
| | SEQ ID | SEQ ID | SEQ ID |
| ELISPOT IFNy | NO: 50 | NO: 45 | NO: 39 |

| | | | | |
|---|---|---|---|---|
| BC303 | Test 1 | − | − | − |
| | Test 2 | − | − | − |
| BC314 | Test 1 | − | − | + |
| | Test 2 | − | − | + |
| BC228 | Test 1 | − | − | − |
| | Test 2 | − | − | − |
| BC320 | Test 1 | − | − | − |
| | Test 2 | − | − | − |
| BC322 | Test 1 | − | − | − |
| | Test 2 | − | − | − |
| BC323 | Test 1 | − | − | − |
| | Test 2 | − | − | − |
| | | NEG | NEG | POS |

1. An immunogenic peptide of 20 to 45 amino acid residues in length comprising one or more cysteine residues, wherein said one or more cysteine residues is present in a modified form wherein said one or more cysteine residue is bound to a cysteine or other thiol-containing compound via a disulfide bond, wherein said disulfide bond is not an intramolecular disulfide bond between two cysteine residues within the same immunogenic peptide molecule and wherein said disulfide bond is also not an intermolecular disulfide bond connecting two immunogenic peptide molecules.

2. The immunogenic peptide according to claim 1, wherein said immunogenic peptide comprises at least one MHC class I ligand.

3. The immunogenic peptide according to claim 1, wherein said immunogenic peptide comprises at least one MHC class II ligand, wherein said immunogenic peptide comprises at least one MHC class I ligand and one MHC class II ligand.

4. The immunogenic peptide according to claim 1, wherein said one or more modified cysteine residues are cystines.

5. The immunogenic peptide according to claim 1, wherein said immunogenic peptide comprises two or more modified cysteine residues, such as three or more modified cysteine residues, for example four or more modified cysteine residues.

6. The immunogenic peptide according to claim 1, wherein said immunogenic peptide is from 26 to 45 amino acid residues in length, such as 26, 27, 28, 29, 30, 31, 32, 33, 34, 35, 36, 37, 38, 39, 40, 41, 42, 43, 44 or 45 amino acids in length.

7. The immunogenic peptide according to claim 1, wherein said immunogenic peptide comprises or consists of a sequence selected from the group of sequences set forth in SEQ ID NO: 1-18 or the group of sequences set forth in SEQ ID NO: 38, 39, 40 and 44.

8. An immunogenic composition comprising an immunogenic peptide according to claim 1.

9. The immunogenic composition according to claim 8, wherein said immunogenic composition comprises a pharmaceutically-acceptable carrier, wherein said immunogenic peptide is present at a concentration of at least 10 microgram/mL.

10. The immunogenic composition according to claim 8, wherein said immunogenic peptide is dissolved in an oxidizing solvent, such as 20% v/v DMSO in water.

11. The immunogenic composition according to claim 8, wherein the composition comprises at least one further immunogenic peptide as defined in any one of the preceding claims.

12. A medicinal product comprising one or more immunogenic peptides according to claim 1.

13. The immunogenic peptide, immunogenic composition or medicinal product according claim 1 for use in medicine.

14. The immunogenic peptide, immunogenic composition or medicinal product according to claim 1 for use in inducing an immune response in a human subject, wherein said immune response comprises a CD8+ T cell response.

15. A method for producing an immunogenic peptide according to claim 1, comprising synthesizing the immunogenic peptide chemically.

16. A medicinal product comprising one or more immunogenic compositions according to claim 8.
